(19) United States
(12) Patent Application Publication
(10) Pub. No.: US 2016/0367747 A1
(43) Pub. Date: Dec. 22, 2016

(54) OPEN-PORE BALLOON CATHETER

(71) Applicant: LOHMANN & RAUSCHER GMBH, SCHOENAU AN DER TRIESTING (AT)

(72) Inventor: GUNNAR LOSKE, AHRENSBURG (DE)

(21) Appl. No.: 15/180,798

(22) Filed: Jun. 13, 2016

(30) Foreign Application Priority Data
Jun. 11, 2015 (EP) 15001736.6

(51) Int. Cl. A61M 3/02; A61M 25/10; A61M 31/00; A61M 1/00; A61B 17/34; A61M 25/09

(52) U.S. Cl. CPC A61M 3/0295; A61B 17/3403; A61B 17/3415; A61M 25/09; A61M 31/00; A61M 1/0084; A61M 3/0283; A61M 1/005; A61M 25/1002; A61B 2017/3413; A61M 2210/101; A61M 2210/1053; A61M 2210/1085; A61M 2205/18; A61M 2025/105; A61M 2025/1088; A61M 2025/09175

(57) ABSTRACT

The invention relates to a catheter, in particular a balloon catheter, having a fluid-carrying element, such as a drainage tube, with an inner lumen for carrying away body fluid, and having an expansion element, such as a balloon of variable cross section, for fixing the catheter in a body cavity. The fluid-carrying element has a wall portion of an open-pore material, which is in fluidic communication with the inner lumen and through which the body fluid can be aspirated into the inner lumen by applying a negative pressure to a proximal end of the fluid-carrying element. The invention furthermore relates to a catheter system having a catheter of the invention and to a vacuum-generating system connected to the catheter.

### CROSS-REFERENCE TO RELATED APPLICATIONS

This application is based on and claims priority to European Patent Application Serial No. 15 001 736.6, filed on Jun. 11, 2015, which is incorporated herein by reference in its entirety.

### STATEMENT REGARDING FEDERALLY SPONSORED RESEARCH OR DEVELOPMENT

Not applicable.

### BACKGROUND OF THE INVENTION

The invention relates to a catheter, in particular a balloon catheter, having a fluid-carrying element with an inner lumen for carrying away body fluids and/or gases from the interior of the body, and having an expansion element, such as a balloon, of variable cross section for keeping or fixing the catheter portion in a body cavity. The fluid-carrying element can have a tube element, such as a drainage tube, and the expansion element can have a balloon.

Such catheters are used among other ways as bladder catheters, which are inserted into the urinary bladder either via the urethra (transurethrally) or the abdominal wall (suprapubically) and serve to draw off urine. Particularly when the bladder catheter is to be used as an indwelling catheter, it is known to locate an expansion element, such as an inflatable balloon whose volume is variable, in the vicinity of the distal end (the end to be located in the body interior) of the catheter in order to prevent dislocation of the catheter. When the catheter is correctly placed and the catheter portion having the balloon is located in a body cavity such as the urinary bladder, the balloon volume is increased in such a way that the balloon anchors itself in the body cavity. The catheter is thus self-retaining.

Defects in the urinary bladder, the renal pelvis, and the organs that drain urine lead to the escape of urine. They occur for instance as a consequence of postoperative complications in these organs. They can be closed surgically. Leaks from the urethra, ureter, or the urinary bladder or renal pelvis are also treated conservatively with the aid of passive urine drainage by gravity. For draining urine, transurethral, suprapubic catheters or kidney fistulas are inserted, and the urine is drained into bags in that way. The urine is intended to take the path of least resistance and be carried by gravity directly into the catheter and not take its course via the leak. Leaks from the ureters can also be carried through small-lumen splinting catheters that span the defect, from the renal pelvis into the urinary bladder or beyond through the urethra. These catheters are partly crimped on their end in pigtail-like fashion.

The drainage catheters or probes described are provided with perforation openings in a distal end portion. Via these perforation openings, the urine, or some other body fluid to be discharged from the body, flows by gravity into the inner lumen of the drainage tube and is discharged to the outside via the drainage tube.

From International Patent Disclosure WO 2013/029622, it is known to apply a negative pressure to the fluid-carrying element in order to accelerate urine drainage. However, it has been found that because the outer wall of the fluid-carrying element contacts an inner wall of the body, accelerated drainage may become more difficult. This disadvantage is overcome by the invention, and numerous novel possibilities of negative pressure treatment for wound treatment are described.

### BRIEF SUMMARY OF THE INVENTION

In view of the described problems, it is the object of the present invention to refine a catheter, anchored in the body by means of an expansion element, in such a way that it can be used for fast, reliable drainage of body fluids by applying negative pressure to the catheter.

This object is attained by a catheter, in particular a balloon catheter, in accordance with claim 1. Advantageous refinements of the invention are described in the dependent claims. The catheter of the invention is distinguished in that the fluid-carrying element has a wall portion of an open-pore material, which is in fluidic communication with the inner lumen. The body fluid can be aspirated into the inner lumen by the application of a negative pressure at a proximal end (the end facing away from the body) of the fluid-carrying element. Advantageously, a distal end portion of the fluid-carrying element is surrounded by the open-pore material. The invention thus furnished an open-pore balloon catheter, to which a negative pressure can be applied.

The invention traces back to the recognition that the perforation openings in conventional drainage catheters easily become clogged, so that it is not ensured that fluid can be carried permanently via the drain line. Moreover, the drainage tube in conventional catheter systems with perforation openings can be pressed by suction against the body tissue when a negative pressure is applied to the tube system. Because of the suction against the body tissue, the perforation openings become closed, so that fluid can no longer be carried into the inner lumen. By comparison, the invention proposes that self-anchoring catheters, such as balloon catheters, be equipped as open-pore drainage, so that a negative pressure can be applied to the catheter with the aid of negative-pressure generating systems. Because of its open-pore nature, it becomes more difficult for the wall portion facing toward the body to be pressed against the tissue by the suction. Moreover, the open-pore construction prevents clogging of the drain and ensures that the drain will carry fluid permanently. Liquids such as urine can thus be actively carried away with negative pressure. With the invention, fast and reliable drainage of secretions by suction is made possible while retaining all the advantages of the passive discharge catheter retained (placement by puncturing; placement via any natural body openings, such as the anus, mouth, nose, urethra, etc.; shaping of the entire drain in the form of a tube; protection against dislocation because of the integration of the expansion element).

Negative pressure therapy (also called vacuum therapy and low pressure therapy) has become established as a treatment method for treating external infected wounds. An open-pore fluid collection means (such as gauze or polyurethane foam) is placed in the wound and occluded with a film, and a vacuum is applied to the wound. The dressing is changed at regular intervals. The wound is self-cleaning, and wound edema and secretions are carried away. A stable wound surface forms, with granulation tissue, resulting in complete wound healing. It has already been possible to demonstrate that the principle of healing external wounds by means of negative pressure therapy can be employed in the same way intracorporeally. With endoscopes, the open-pore dressing material, to which a vacuum-generating system can be connected, can be placed intracorporeally in body cavities via natural or artificial body openings. Typical treatment indications are leaks in the gastrointestinal tract, especially in the rectum and esophagus. The treatments are employed intracorporeally and in intra-cavity fashion in a wound cavity, and intraluminally directly in the intestinal lumina. Moreover, negative pressure therapy is already being used for peritoneal infections. A double-layer drainage film (Suprasorb® and CNP Drainage) with open-pore drainage properties can be placed directly on the intestine for this purpose. The suction is carried onward to the drainage film underneath the occlusion foil, through open-pore polyurethane foam or gauze.

Until now, it was problematic to apply negative pressure therapy for leaks in the urinary organs, the urinary bladder or the renal pelvic calyx system. It has now been found that because of its open-pore design, which prevents it from being sucked against the tissue and yet permanently maintains the capability of suction, a balloon catheter of the invention with a drainage tube can be used especially advantageously for intracorporeal applications of negative pressure therapy, especially in the region of body cavities, such as the urinary bladder.

The invention can be used in the human and the animal body. By means of the negative pressure, liquids and gases can be actively drained off. Urine, in particular, can be drained off All liquid intracorporeal fluids can be drained, such as seromas, lymph, hematomas, abscesses, ascites, bile, pus, blood, gastric juice, small intestine secretion, pancreatic secretion, pleural fluids, lymph, and gases, among others.

The fluid-carrying element and fluid communication element of the catheter of the invention advantageously has a negative pressure-stable tube, which does not collapse even with the application of a negative pressure. The drained gases and liquids flow in the tube. The tube has at least one fluid carrying inner lumen. On the proximal end, the tube can be connected fluidically via a connecting element to a negative-pressure generating system, such as a pump system, which is equipped with a secretion collecting container in which the drained secretion is caught. The wall portion of open-pore material is preferably located in a distal end portion of the tube. In particular, the tube wall surrounding the inner lumen in a closed fashion can merge at its distal end with the open-pore wall portion.

The open-pore material preferably has a plurality of pores that communicate fluidically with one another, so that the individual perforations to the inner lumen also communicate fluidically with one another. The pores can be located directly beside one another, as in an open-pore polyurethane foam. They can also be spaced apart from one another. Under negative pressure, the open-pore fluid-carrying capability for liquids and gases is preserved. The open-pore wall portion communicates fluidically with the inner lumen of the fluid-carrying element or tube. In a distinction from the lateral perforation openings of a conventional drainage tube, which when subjected to a negative pressure are aspirated against the wall of a body cavity or an organ and stop up, so that no further fluid carrying takes place, in the case of a drainage tube equipped with open pores, the fluid is carried via the multiply intercommunicating pore openings, even under negative pressure. For instance, when a wall portion arranged in this way is in suprapubic contact with the bladder, urine can be permanently and actively drained from the bladder over the long term. The bladder collapses partially or completely around the distal catheter end that has the open-pore wall portion. In this way, in the event of leakage from the bladder, urine is prevented from entering into a defect of the bladder. During the long-term drainage, the bladder defect can heal.

The pores preferably have an opening diameter between 100 μm and 2000 μm, in particular between 100 μm and 500 μm. They can be located immediately adjacent one another or spaced apart from one another. The mean spacing of two most closely adjacent pores is preferably between 100 μm and 5000 μm, in particular between 100 μm and 1000 μm.

In special forms of the catheter of the invention, the preservation of the principle of having open pores is maintained by means of intercommunicating slits in and/or on the wall of the fluid-carrying element. The description of pores does not necessarily circumscribe a circular or round opening of a communicating volume. Pores may also be slitlike, elongated, ellipsoid, tubelike, etc.

The open-pore nature in the vicinity of the distal end of the catheter can be achieved in various ways. With regard to the fluid withdrawal properties, it is especially advantageous to use an open-pore foam, such as an open-pore polyurethane foam or an open-pore single-, double-, or multi-layer film, such as the double-layer drainage film mentioned at the outset.

In a first preferred embodiment of the invention, the inner lumen in a distal end portion of the fluid-carrying element is bounded by a wall that has (perforation) openings. These wall openings can be covered by the open-pore material. Fluid is carried from the outer surface of the open-pore material into the interior of the material as far as the inner lumen of the fluid-carrying element.

In an alternative embodiment, the inner lumen bounded at least in some portions directly by the open-pore material. The open-pore material can embody the wall surrounding a distal end portion of a drainage tube and/or can adjoin a distal end of a non-open-pore tube wall. If the wall of the fluid-carrying element is already intrinsically constructed with open pores, at least in some portions, it is provided with countless pores communicating with one another, so that the fluid is carried from the outside of the wall, through the wall, to the inner lumen and/or inside it and along the wall.

The open-pore material preferably has open pores on one side, and the open-pore side for contact with a body tissue faces outward, and the inner side opposite from the open-pore side faces toward the inner lumen and can have apertures.

The material that has open pores on side may be a polyurethane foam with open pores on one side and/or a film with open pores on one side. The open-pore side of the film or foam is located on the outside, and the closed side is located inward toward the drainage tube or toward the inner lumen. Via openings in the tube wall, the closed side can be perforated, so that fluid is carried, from the outer face of the material that has open pores on one side, into the interior of the material, as far as the inner lumen of the fluid-carrying element. The material with open pores on one side drains the body fluid along and inside the wall of the fluid-carrying element. The material with open pores on one side may optionally be drained via separate suction channels. The fluid-carrying element may also have two or more inner lumina, which are used for draining the open-pore material.

In certain embodiments, the catheter of the invention is constructed such that the fluid-carrying element in one or more drain portions extending along the main axis of the catheter comprises entirely open-pore material, and no inner lumen extending along the main axis of the catheter, on the order of a tube or a centrally extending channel, is provided. At least a first drain portion comprising purely open-pore material can be provided on the distal end of the catheter, and/or at least one second drain portion comprising entirely open-pore material can be provided in a middle catheter portion, proximally and/or distally of the expansion element. A central inner lumen, which is arranged for carrying away or carrying body fluids and/or gases further, of at least one open-pore drain portion can adjoin the proximal and/or distal end. In other words, the catheter of the invention does not necessarily have a drainage tube with an inner lumen in all sectional planes.

After the suction is applied to the proximal end of the inner lumen, the fluid can be carried inside the fluid-carrying open-pore drain portions. In particular, it should be possible for the special form of the drain to be equipped with a guide wire, which can be passed into the inner lumen of the tube and through the open-pore drain portion. This form of catheter can be produced with a very small outer diameter, yet its fluid-carrying properties still remain preserved. They are especially well suited for placement in small lumina (ureter, bile ducts, pancreatic ducts, fistulas).

The tensile and thrusting strength of the open-pore wall portion can be increased by means of reinforced threads, wires or the like, extending longitudinally at the catheter, that are incorporated into a wall of the fluid-carrying element in these portions, and/or proximally and/or distally into a wall of the catheter. The placement can be done using radiological monitoring. Placement in the working channel of endoscopes is possible. The outside diameter of the fluid-carrying element in this embodiment is in particular 1-5 mm, and/or the catheter length may be between 10 cm and 150 cm.

In some embodiments, the catheter of the invention can be curved in pigtail-like fashion on the distal end, in order to anchor itself with the curvature in a cavity (such as an abscess cavity or a hollow organ).

The drain can also be curved in pigtail-like fashion on both ends. In this case, it can be anchored by its distal or its proximal end in a cavity (for instance as a ureter drain in the renal pelvis and the bladder). This kind of special form may also be used as passive drainage, without applying negative pressure. The advantage of this embodiment of the invention is that the fluid can be carried in all portions of the open-pore wall portion of the catheter, and the liquid can be collected over the entire surface of the open-pore wall portion.

If the open-pore material has one or more films, they are preferably nontransparent, in particular being embodied in color or dyed. The dyed films serve to provide security in endoscopic monitoring of internal wound surfaces. If a film should be aspirated so firmly against an internal wound that, in a maneuver for removing it, it tears off from the fluid-carrying element entirely or partially, then in the case of the transparent embodiment it is difficult to distinguish the foreign material from the tissue. In a colored version, the foreign material can easily be discovered endoscopically. Advantageously, the film is also radiopaque. Alternatively, however, a transparent foil may also be used.

The wall portion of open-pore material is preferably located in a distal end portion of the fluid-carrying element and there can at least partly and preferably completely surround the inner lumen there and/or demarcate it distally. The wall portion thus at least in some portions forms an outer layer of the side wall of a distal catheter portion. Alternatively or in addition, the wall portion of open-pore material can also embody the distal end of the catheter, that is, its forward front wall.

The expansion element preferably has a balloon with an elastically stretchable wall that at least partly bounds a fillable volume of the balloon. Filling the fillable volume of the balloon inflates the balloon, increasing its cross section, thereby making it possible to anchor the catheter in a body cavity in a way that is especially gentle to the tissue. The balloon wall may be formed of a flexible stretchable cloth, such as stretchable plastic. Instead of the balloon, it is expressly also possible to use other expanding locking mechanisms around or on the drainage tube. They may for instance comprise wall portions that open in umbrellalike fashion or spread out and are closable again, or they may comprise a counterpressure plate of the tube wall.

A channel that carries fluid and/or gas may be provided for filling and/or emptying the fillable volume, the channel extending essentially parallel to the fluid-carrying element. The channel may at least in some portions extend in the inner lumen of the fluid-carrying element, outside the inner lumen of the fluid-carrying element, in, or in contact with, a side wall of the fluid-carrying element in its longitudinal direction, or alternatively extend as a separate tube, spaced apart from the fluid-carrying element.

In a first embodiment of the invention, the channel extends longitudinally of the fluid-carrying element and in particular in its outer wall as an additional gastight and/or watertight lumen. For example, the channel extends in the tube wall of the drainage tube. In a second embodiment of the invention, the channel is spaced apart from the drainage tube and is located outside the inner lumen. For instance, the channel is formed as an additional tube. In a third embodiment of the invention, the channel extends as a tube located inside the inner lumen of the fluid-carrying element. If the drainage tube is equipped with more than one fluid-carrying inner lumen, one of the lumina can serve as a channel for filling the balloon and the other can serve to carry away the body fluid.

By means of a valve on the proximal end of the channel, it is possible to prevent the introduced gas or the liquid from escaping, and it can be ensured that the balloon will stay pumped up for a long time. The gas or the liquid can be removed from the balloon via the balloon via the valve. The gas or liquid cannot escape or can escape only slowly from the fillable volume, so that the fill state of the balloon needs to be checked only infrequently (less than once a day).

The balloon preferably has a volume of 0 to 200 ml. The balloon surrounds the circumference of the fluid-carrying element at least in part, preferably completely, and/or in the inflated state is annular and/or essentially cylindrical. Alternatively, in the inflated state, the balloon can be spherical and can form the distal end of the catheter.

Alternatively, the balloon can surround the catheter only in some portions and not all the way around. Also alternatively, the catheter of the invention may have more than one expansion element, such as two or more balloons.

One or more further channels may be provided for the purpose of rinsing, delivering medications, measuring pressure, and/or passive drainage. A rinsing channel of this kind is open on its distal end. Via the rinsing channel, the cavity to be drained (urinary bladder, abscess cavity) can be supplied with liquids or medications or can be flushed. Via at least one further channel, measuring instruments, for instance for controlling the vacuum-generating unit and for measuring negative pressure, can be delivered, or other kinds of measurements may be made, via the lumen of the channel. The catheter is also intended to be usable for suction rinsing treatment. For instance, after insufficient rectal anastomosis, it can be used to continue wound treatment with a rinsing treatment. The catheter would then be introduced rectally to the rinsing site, rinsed via the one channel, and with the other channel the rinsing secretion can be drained out, actively by means of suction, or passively. The draining channel is constructed with open pores and can optionally additionally be provided with perforation openings on the distal channel end.

In a preferred embodiment of the invention, the balloon widens by insufflation of gas or liquid via the channel entirely or partly annularly around the fluid-carrying element or spherically on the distal end of the catheter. The balloon may be embodied as part of a side wall of the fluid-carrying element; the wall in that portion is arranged to be flexible and elastically, so that the balloon can be filled with liquid and/or gas.

In a special embodiment of the invention, the fillable volume of the balloon is filled at least partially by a filling material, such as an open-pore polyurethane foam, which contracts against the fillable volume when a negative pressure is applied and when the negative pressure is withdrawn causes the balloon to unfurl. This function has the advantage that the balloon, with the filler material, can adapt to the cavity that is to be relieved. The filler material may take various forms. For instance, such a balloon can assume the shape of a sphere or cylinder. The spherical form should in particular have a diameter of 1 cm to 10 cm. The cylindrical form should in particular have a diameter of 1 to 5 cm and a length of 1 cm to 20 cm. A negative pressure can be applied to the balloon so that it collapses into itself completely. This function can make it easier to place a balloon drain (for instance when changing the drain).

In view of good, reliable placement of the open-pore material, it has proved expedient that the wall portion of the open-pore material directly adjoins the balloon distally. However, it is also possible for the balloon itself to form the distal end of the catheter and for the wall portion of the open-pore material to be located at least in some portions outside on the elastically stretchable wall of the balloon.

In a preferred embodiment of the invention, the wall portion of open-pore material is located on a distal end portion of the fluid-carrying element. Alternatively or in addition, it is possible for a wall portion of open-pore material to be provided in a middle portion of the catheter. The expansion element can be located spaced apart from the open-pore wall portion. For instance, a catheter thus equipped can be used for sealing off a ureter suture or leak with the open-pore wall portion located in the middle portion of the catheter, while the expansion element can be anchored in the renal pelvis.

In one possible embodiment of the invention, the wall portions of open-pore material are located distally and/or proximally of the expansion element. The wall portion or wall portions with open pores preferably merge continuously smoothly with non-open-pore wall portions of the catheter in order to facilitate atraumatic insertion. A catheter should be able to have a plurality of open-pore wall portions, which may be located adjacent to one another or spaced apart from one another.

The drainage tube of the fluid-carrying element preferably has a diameter between 2 mm and 25 mm. Alternatively or in addition, the part of the catheter that is insertable into the body has a length between 15 cm and 150 cm. Alternatively or in addition, the catheter in the vicinity of the inflated balloon has a total diameter between 5 mm and 50 mm. The cross section of the catheter can be increased by a factor of approximately 2 or more by expansion of the expansion element.

The open-pore wall portion preferably has a length of more than 1 cm and less than 15 cm.

Simple placement of the catheter in the body is possible because its distal end is closed and/or can have a fastening element, such as an eyelet, thread, ball or the like, for attaching a gripping or tugging element. The closed distal end may be embodied conically or may taper to a point, with the securing element mounted on the tip. With this kind of equipment, the catheter can be placed in body cavities by the pull-through technique by pulling on the tip. With the closed distal end, the drainage tube can be inserted especially atraumatically into the body through natural body openings (such as the urethra) or existing other openings (such as fistula openings, suprapubic stoma to the bladder, or the like). If after the insertion with the tip of the drainage tube the cavity to be drained (such as the bladder or renal pelvis) is reached, the balloon is filled and blocked. By means of the filled balloon, dislocation of the catheter is prevented.

In the suprapubic placement of an open-pore balloon catheter, the balloon is also intended to serve to pull the bladder with the catheter against the peritoneum. At least at the beginning of therapy, the balloon catheter should be pulled against the peritoneum with slight tension. Pulling the wall of the hollow organ against the peritoneum serves in particular to cause the hollow organ to securely adhere to the peritoneum and to create a fistular channel. This is especially important, since as a rule in the placement of the catheter, the hollow organ is full and collapses after the vacuum is applied, and therefore external and internal puncture locations can slide apart. The tension is maintained by fixing the catheter to the skin via the cutaneous entry opening. This fixation with tension can be done using a surgical fixation suture and/or a counterpressure plate in which the catheter is clamped under tension. Once a stable channel around the catheter has formed, the tension can be loosened or withdrawn, since otherwise the mucous membrane of the bladder can be damaged by pressure. In analogy to this description of the manipulation for the bladder, the situation is similar for use with other hollow organs, such as a percutaneous stomach puncture for relieving the stomach.

In a further embodiment of the invention, the catheter has an opening on its distal end, through which a guide wire or guide stylet can be introduced. The guide wire can be equipped atraumatically, flexibly and soft on the distal end. With this kind of wire, the catheter can be introduced into the body for instance using the Seldinger technique.

The guide wire or guide stylet can have a cutting edge or tip for puncturing tissue on its distal end. By its distal end, the drainage tube should merge flush and smoothly with the guide stylet. With this kind of equipment, the catheter can be inserted into body cavities by the puncturing technique. For instance, it is possible to puncture abcesses percutaneously, to puncture the bladder suprapublically, to puncture the renal pelvis percutaneously, and to drain pleural effusions or other reachable accumulations of liquid. The puncturing can be done with sonographic, radiological, computed tomography and/or endoscopic visual monitoring. In suprapubic puncturing, the catheter can also be placed via the lumen of a puncturing tube, which is divisible along the longitudinal axis and is sharp on the distal end, like a suprapubic bladder catheter.

The catheter of the invention can be extended in length and extend straight in the longitudinal axis at its distal end. For other applications, the catheter can be curved in arclike fashion on its distal end longitudinally. For still other applications, the catheter can be crimped in pigtail-like fashion on its distal end longitudinally. Even if the catheter does not extend rectilinearly straight, it can still extend rectilinearly straight via a guide wire.

For fast drainage of body fluid even without applying a negative pressure, it may be advantageous to equip the fluid-carrying element on its distal end with at least one lateral perforation opening, which is not covered by the open-pore material. Via these openings, liquids or gases can be drained by capillary action or gravity. These openings are preferably located distally of the wall portion of an open-pore material.

The open-pore portion of the fluid-carrying element can merge smoothly with a tubelike portion. The diameter of the fluid-carrying element can be essentially the same in both portions. In the open-pore portion, the diameter of the fluid-carrying element should be only insubstantially greater or lesser than the diameter of the closed tube portion. This is intended in particular to ensure easy placement and removal of the catheter, for instance in transurethral placement. In particular, the drain can also be surgically placed in the abdomen, in an abscess, or in the chest for postoperative drainage. Until now, passive drains have conventionally been used for this. The invention has the advantage of putting an active negative pressure drain in place which is capable of becoming anchored via a balloon and cannot become dislocated, to which active suction can be applied, and which can be removed postoperatively, without a further operation, simply by pulling on the unblocked drain.

The catheter of the invention can be used for simultaneous or intermittent rinsing of intracorporeal spaces by suction.

The open-pore wall portion of the fluid-carrying element is preferably located distally of the balloon.

Alternatively or in addition, the open-pore wall portion of the fluid-carrying element is located proximally of the expansion element. In that case, the expansion element can form the distal termination of the fluid-carrying element. A catheter embodied in this way can in particular also be placed as a postoperative drain, for instance intraabdominally, at the conclusion of the operation. The expansion element anchors the drain internally where it is placed. The drain can be employed both as a passive drain and an active drain with the application of a negative pressure. A further typical example of use for such a drain is wound drainage or abscess drainage.

It is expressly also conceived of that a wall surrounding the balloon be additionally embodied in open-pore fashion and/or with open pores on only one side, so that on the balloon-holding wall portion, with the balloon unfurled, incompletely unfurled, or nor unfurled, a negative pressure can be applied. Special forms of the negative pressure drain comprise a catheter which on its distal end has a balloon with an open-pore wall suitable for fluid carrying, to which wall a negative pressure can be applied. Such a catheter is equipped with at least two lumina; one lumen serves to expand the balloon, and the other lumen serves to drain the fluid with application of the vacuum. One advantage of this special drain is its atraumatic position in a hollow organ. No ends of the tube or tips which under the influence of suction could also cause pressure damage to the organ wall protrude past the balloon. The open-pore and/or unilaterally open-pore sheathing of the balloon can be solidly connected to the balloon wall. The open-pore and/or unilaterally open-pore sheathing can also lie loosely, like a bag or jacket around the balloon. The open-pore sheathing is elastic and adapts to the balloon. The catheter can also be used in particular for operative drainage after abdominal operations.

In a further aspect, the invention relates to a catheter system with a catheter of the invention and with a negative-pressure generating system, such as an electronic pump system, which for generating suction in the inner lumen of the fluid-carrying element can be connected to the proximal end of the fluid-carrying element.

At least one negative pressure-stable fluid collection container for catching the body fluid can be connected between the negative-pressure generating system and the fluid-carrying element; this container is advantageously equipped with a valve that prevents a return flow of body fluid into the fluid-carrying element.

The negative-pressure generating system should be arranged for generating a permanent negative pressure between approximately 10 mmHg and approximately 200 mmHg, and in particular between approximately 30 mmHg and approximately 100 mmHg.

If only small quantities of body fluid are to be drained, a container volume between 100 ml and 500 ml suffices. Larger containers can also be connected to the pump system. In particular in the case of urine drainage, for which the invention is particularly well suited, even larger quantities of body fluid can be drained. Here, a further negative pressure-stable collection container, which may be equipped with the valve, can be placed between the catheter and the fluid collection container. This makes it possible to catch quantities of between 500 ml and 2500 ml. That has the advantage that the vacuum-generating pump (pump with a small container for generating a vacuum upstream) can be kept relatively compact and small. If very large collection containers are used on the pump itself, then the pump becomes very unwieldy, and the patient is made immobile.

The electronic pump is intended to be preferably small in size. This is especially possible if a high negative pressure and a high suction volume per minute are not necessary. Such a pump should be used for instance for active urine drainage from the renal pelvis and/or from the bladder. Here, because of the intracorporeal position with the transurethral or suprapubic placement (unlike in therapy of external wounds with occlusion dressings), leaks at a dressing need not be expected.

In this application, the pump should be able to drain the urine volume that occurs. In the normal case in healthy adults, this is between 1.5 and 2 liters per 24 hours. Depending on the amount a person drinks (or infusion quantity) or because of illness, the urine quantity can even be more than 2 liters a day. Since urine production occurs continuously, with certain fluctuations, the pump for urine production of 2 liters in 24 hours must be capable of draining at least approximately 100 ml per hour on average. The pump should be capable of draining 500 ml of fluid per hour. Since the natural urine flow is carried into the bladder by capillaries and muscular contractions of the ureter, and since with passive drainage or leakage the urine flows by gravity, the pump must be capable at least of draining counter to the force of gravity. If the vacuum-generating system is at the same level as the bladder to be drained, then a suction of only a few centimeters of water column already suffices for drainage. If the vacuum-generating system is located above the site to be drained, then first this height difference has to be overcome. The situation is analogous for the reverse case, if the pump is placed below that level.

Regardless of the position of the body fluid to be drained, the pump should therefore be capable of applying a negative pressure of at least 20 mmHg to the fluid-carrying element within one minute. This suction should be applied permanently to the bladder. It can be permanently reregulated by means of a controller. The pump should be capable, for use in the bladder, of preferably generating a negative pressure of 20 mmHg to 100 mmHg.

In a first embodiment of the invention, the pump generates its vacuum via a upstream container. In an alternative embodiment of the invention, the pump generates the vacuum without a upstream container. The pump is preferably arranged such that it can be made to communicate fluidically with a fluid collection container that has a volume of 250 to 2000 ml. If the pump has an upstream container, then that can be integrated with the fluid collection container. A pump to be used for urine drainage should not be larger than approximately 5 cm×10 cm×20 cm.

The manipulation of the system of the invention can be simplified by providing that the pump can be operated with a rechargeable battery. Alternatively or in addition, the pump can be connected to the fluid collection container by a releasable locking mechanism. Transporting the pump is simplified by providing that the pump is a unit including the one or more fluid collection containers, such as a bag or canister system. The pump can accordingly be part of a bag/canister system for collecting body fluid. To that end, the unit comprising the fluid collection container and the pump can be provided with a carrying handle and/or strap.

The manipulation is especially advantageous if the secretion or body fluid is drained into a bag system. The bag system must be capable of taking on the suction and carrying it onward. This can be achieved if the bag system has a filler material, such as an open-pore and/or compressible fluid collection element (such as an open-pore polyurethane foam). In the lumen used, the filler material does not collapse, or collapses only incompletely, and keeps the lumen of the bag open for receiving secretion while maintaining suction. The advantage of the bag system is also the small volume it requires in shipping and storage. By evacuating the bag system before use with a high vacuum or by compression, very small, space-saving packaging units can be produced.

By dividing the bag system into chambers, incremental filling can be done chamber by chamber. The urine can also be introduced into one bag portion that is not equipped with filler material.

Moreover, a combination of a bag system and a solid canister container can be conceived of for the fluid collection container. The canister container can function as an upstream volume for the pump.

The bags can also be used for draining secretions in abdominal negative pressure therapy with drainage films, for pleural drainage, or for active stomach relief. Often in such situations, the secretion lumina are relatively large.

The collection container, such as the bag and/or the canister, is preferably provided with measurement markings for determining volume, in order to be able to balance the quantity of secretion.

The catheter of the invention can be provided with a marking (preferably in centimeters) to simplify manipulation, so that its insertion depth can be read off and any dislocation can be found. Alternatively or in addition, a radiological marking may be provided.

Treatment is done with a negative pressure between 10 and 200 mmHg, preferably with a negative pressure between approximately 40 and 200 mmHg. The drainage can be done continuously or intermittently. In urine drainage, negative pressures between 20 and 100 mmHg are employed in particular. The pump can be equipped with a controlling and alarm function for pressure control. As a result, drain blockages, drain leaks, and the status of the canister and/or bag filling can be recorded and reported to the control unit.

As examples, two typical examples of use for the invention follow.

### EXAMPLE 1

In an operation on the rectum, an injury to the back wall of the bladder occurs and is sutured. The bladder is supplied intraoperatively with a conventional transurethral catheter and a suprapubic catheter for draining urine. In the postoperative course, urine emerges via the surgical drain. Since the urine drainage via the passive catheter already in place was inadequate, the urine was evacuated via the incompletely closed defect in the abdominal cavity and came to be connected to the surgical drain. On the expectation that the escape of urine could be avoided by draining urine directly from the kidneys and drying the urinary bladder, ureter splinting catheters are placed transurethrally in both renal pelvises via both ureters. Here again, urine drainage does not take place via the passive drains; urine draining persists into the surgical drain.

Now, the treatment with the invention follows: The suprapubic catheter is replaced, using the Seldinger technique, with a catheter of the invention in the form of an open-pore balloon drain; the balloon is blocked and, with an electric pump, a continuous permanent suction of 80 mmHg is applied to the open-pore distal end of the drain. Immediately after the negative pressure is applied, the urinary bladder collapses, and all the urine still remaining in the bladder is aspirated completely. As a consequence, the urine secretion via the surgical drain in the transurethral catheter is suspended. Unlike with the use of conventional catheters, it is now possible, with the bladder collapsed, to drain off nearly all the urine production via the two transurethral kidney fistulas. After several days, the open-pore balloon drain is changed a single time by the Seldinger technique via a guide wire. In the process, the urinary bladder and the internal wound are inspected endoscopically, and the course of healing of the internal wound is monitored. After 18 days of urine drainage, after the urinary bladder defect has healed, the open-pore drain therapy can be discontinued. The healing of the defect was confirmed beforehand both radiologically and endoscopically. All the transurethral catheters and the surgical drain or drains are removed. The application of negative pressure to the open-pore drain is ended, and the drain is left in the urinary bladder for several days for passive urine drainage and for training the bladder. Once the bladder has been trained with passive urine drainage, the catheter of the invention, as the final drain, is removed.

### EXAMPLE 2

A female patient has developed a postoperative seroma, which can easily be reached by puncturing during sonographic monitoring. After being located sonographically and after local anesthesia is administered, the puncturing is done with a catheter of the invention in the form of a balloon drain, which is equipped with a sharp guide stylet. The tip of the catheter is placed in the seroma cavity; the balloon is blocked, and the catheter is fixed in the cavity. With an electronic pump system, a negative pressure of 80 mmHg is applied with continuous suction to the open-pore distal end. The cavity of the seroma is actively aspirated completely and permanently; the cavity collapses around the drain. The suction is maintained for a total of 3 days. The sonographic monitoring confirms the complete drainage of the seroma, and the drain can be removed.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

In the description that now follows, the invention is described in examples in conjunction with the appended drawings. The invention is not limited to the embodiments shown in the drawings. In the drawings:

FIG. 1a shows a first embodiment of a balloon catheter of the invention in a side view;

FIG. 1b shows the first embodiment, shown in FIG. 1a, in a longitudinal sectional view;

FIG. 1c shows a second embodiment of a balloon catheter of the invention in a side view;

FIG. 1d shows the second embodiment, shown in FIG. 1c, in a longitudinal sectional view;

FIG. 1e shows a third embodiment of a balloon catheter of the invention in a side view;

FIG. 1f shows the third embodiment, shown in FIG. 1e, in a longitudinal sectional view;

FIG. 2a shows a fourth embodiment of a balloon catheter of the invention in a side view;

FIG. 2b shows the fourth embodiment, shown in FIG. 2a, in a longitudinal sectional view;

FIG. 3a shows a fifth embodiment of a balloon catheter of the invention in a side view;

FIG. 3b shows the fifth embodiment, shown in FIG. 3a, in a longitudinal sectional view;

FIG. 3c shows a wall portion of a fluid-carrying element of a sixth embodiment of a balloon catheter of the invention in a side view;

FIG. 3d is a detail view of FIG. 3c in a longitudinal sectional view;

FIG. 3e shows a wall portion of a fluid-carrying element of a seventh embodiment of a balloon catheter of the invention in a side view;

FIG. 3f shows the embodiment, shown in FIG. 3e, in a longitudinal sectional view;

FIG. 3g shows an eighth embodiment of a balloon catheter of the invention in a side view;

FIG. 3h shows the embodiment, shown in FIG. 3g, in a longitudinal sectional view;

FIG. 4a shows a ninth embodiment of a balloon catheter of the invention in a side view;

FIG. 4b shows the ninth embodiment, shown in FIG. 4a, in a longitudinal sectional view, with the balloon not inflated;

FIG. 4c shows the ninth embodiment, shown in FIG. 4a, in a longitudinal sectional view, with the balloon inflated;

FIG. 5a shows a tenth embodiment of a balloon catheter of the invention in a longitudinal sectional view, with the balloon not inflated;

FIG. 5b shows the tenth embodiment, shown in FIG. 5a, in a longitudinal sectional view, with the balloon inflated;

FIG. 6a/b shows a first embodiment of a catheter system of the invention in a side view and in a sectional view;

FIG. 7a/b shows a second embodiment of a catheter system of the invention in a side view and in a sectional view;

FIG. 8a/b shows a third embodiment of a catheter system of the invention in a side view and in a sectional view; and

FIG. 9a/b shows a fourth embodiment of a catheter system of the invention in a side view and in a sectional view.

### DETAILED DESCRIPTION OF PREFERRED EMBODIMENT

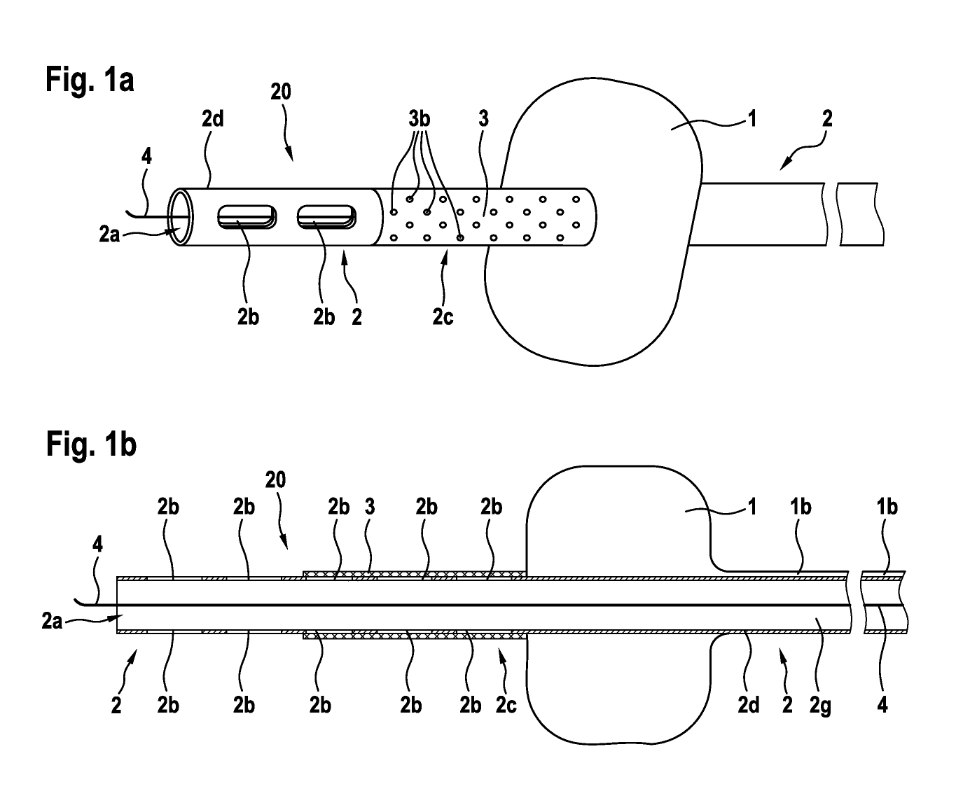

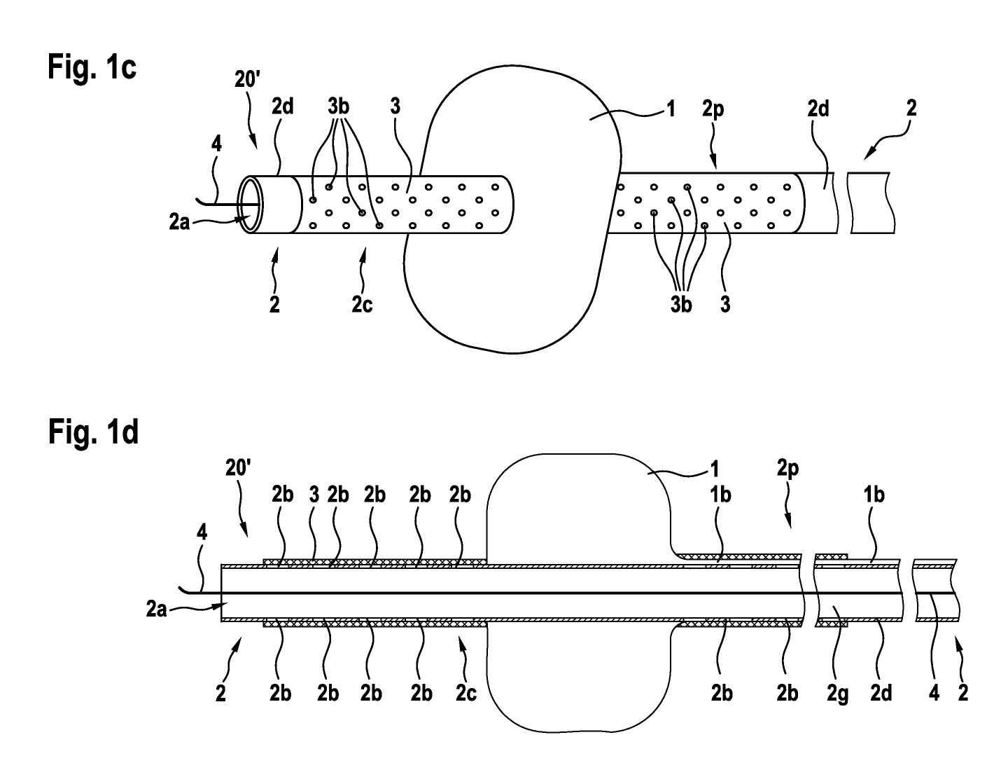

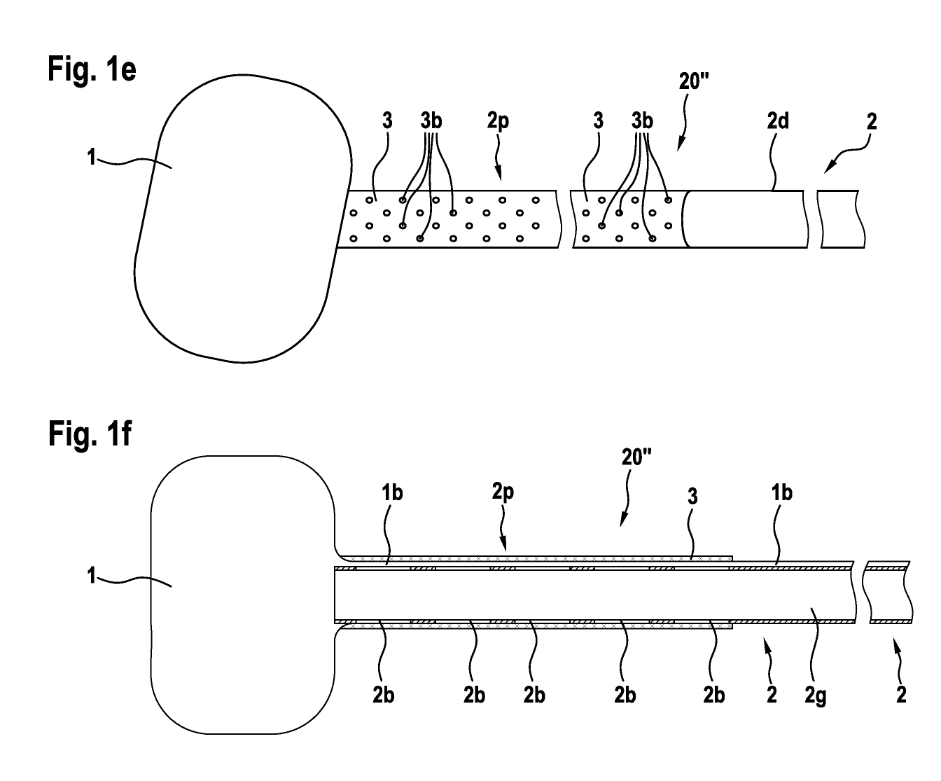

In FIGS. 1a and 1b, a first embodiment of a catheter 20 of the invention, in the form of a balloon drain, is shown in a side view (FIG. 1a) and in a longitudinal sectional view (FIG. 1b).

The balloon drain is shown with an expansion element in the form of an expanding balloon 1, the cross section of which is enlarged in order to firmly hold the distal end of the catheter in a body cavity. The balloon 1 is seated annularly on a fluid-carrying element 2, which has a drainage tube 2d with an inner lumen 2g and extends from the proximal end, which emerges from the body and is shown on the right in the drawings, in the direction of the distal end of the catheter, which is to be located in the interior of the body and is shown on the left in the drawings. The drawings essentially show only the distal end portion of the catheter; the length of the drainage tube 2d is shown shorter than it actually is.

Distally of the balloon 1, the drainage tube 2d is circularly sheathed by a wall portion 2c of an open-pore material 3, in the form of an open-pore film with multiple pore openings 3b.

The drainage tube 2d has a distal tube opening 2a and lateral perforation openings 2b; at least some of the lateral perforation openings 2b are covered by the open-pore material 3. The open-pore material 3 surrounds part of the distal end portion of the drainage tube completely. A guide wire 4 protrudes from the distal tube opening 2a.

FIG. 1b is a longitudinal sectional view of the first embodiment. The expanded balloon 1 can be seen, which communicates fluidically with a channel 1b for filling the balloon 1. The channel 1b is located in the wall of the drainage tube 2d. Distally of the balloon 1, the drainage tube 2d is sheathed by the open-pore film. Moreover, the lateral perforation openings 2b in the tube wall are shown, some of which are covered by the open-pore material 3. The guide wire 4 extending in the inner lumen 2g through the fluid-carrying element 2 protrudes from the distal opening 2a of the tube.

In FIGS. 1c and 1d, a second embodiment of a catheter 20′ of the invention in the form of a balloon drain is shown in a side view (FIG. 1c) and in a longitudinal sectional view (FIG. 1d).

The balloon drain is shown with an expansion element in the form of an expanded balloon 1, whose cross section is enlarged in order to firmly hold the distal catheter end in a body cavity. The balloon 1 is seated annularly on a fluid-carrying element 2, which has a drainage tube 2d with an inner lumen 2g and extends from the proximal end of the catheter, which protrudes from the body and is shown on the right in the drawing, in the direction of the distal end, which is to be located in the interior of the body and is shown on the left in the drawings. The drawings essentially show only the distal end portion of the catheter; the length of the drainage tube 2d is shown shorter than it actually is.

Distally of the balloon 1, the drainage tube 2d is sheathed circularly by a first wall portion 2c of an open-pore material 3, in the form of an open-pore film with multiple pore openings 3b. Proximally of the balloon 1, the drainage tube is circularly sheathed by a second wall portion 2p of an open-pore material 3, in the form of an open-pore film with multiple pore openings 3b.

The drainage tube 2d has a distal tube opening 2a and lateral perforation openings 2b; at least some of the lateral perforation openings 2b are covered by the open-pore material 3. The open-pore material 3 completely surrounds part of the distal end portion of the drainage tube 2d. A guide wire 4 protrudes from the distal tube opening 2a. The open-pore material 3 is located distally (2c) and proximally (2p) of the expanded balloon 1.

FIG. 1d is a longitudinal sectional view of the second embodiment. The expanded balloon 1 can be seen, which communicates fluidically with a channel 1b for filling the balloon 1. The channel 1b is located in the wall of the drainage tube 2d. Distally (2c) and proximally (2p) of the balloon 1, the drainage tube 2d is sheathed by the open-pore material 3. Also, the lateral perforation openings 2b are shown in the tube wall; some of them are covered by the open-pore material 3. The guide wire 4 extending in the inner lumen 2g through the fluid-carrying element 2 protrudes from the distal opening 2a of the tube.

In FIGS. 1e and 1f, a third embodiment of a catheter 20″of the invention is shown in the form of a balloon drain, in a side view (FIG. 1e) and in a longitudinal sectional view (FIG. 1f).

The balloon drain is shown with an expansion element in the form of an expanded balloon 1, whose cross section is enlarged in order to firmly hold the distal catheter end in a body cavity. The balloon 1 is seated annularly on a fluid-carrying element 2, which has a drainage tube 2d with an inner lumen 2g that extends from the proximal end of the catheter that protrudes from the body and is shown on the right in the drawing, in the direction of the distal end, to be located in the interior of the body and shown on the left in the drawings. The drawings essentially show only the distal end portion of the catheter; the length of the drainage tube is shown shorter than it actually is.

Proximally of the balloon 1, the drainage tube 2d is circularly sheathed by a wall portion 2p of an open-pore material 3, in the form of an open-pore film with multiple pore openings 3b.

The drainage tube 2d ends distally with the balloon 1 and proximally of the balloon has lateral perforation openings 2b; at least some of the lateral perforation openings 2b are covered by the open-pore material 3. The open-pore material 3 completely surrounds part of the distal end portion of the drainage tube 2d.

FIG. 1f is a longitudinal sectional view of the third embodiment. The expanded balloon 1 can be seen, which communicates fluidically with a channel 1b for filling the balloon 1. The channel 1b is located in the wall of the drainage tube 2d. Proximally (2p) of the balloon 1, the drainage tube 2d is sheathed by the open-pore film. Also, the lateral perforation openings 2b in the tube wall proximally of the balloon are shown, which are covered by the open-pore material 3.

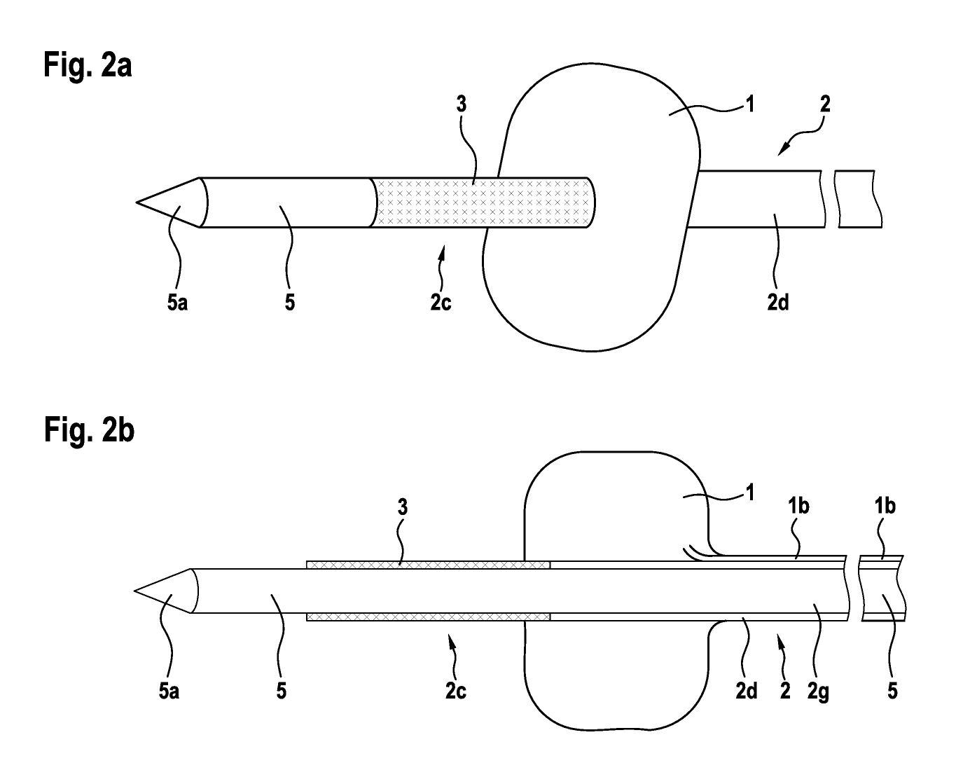

FIG. 2a is a side view of a fourth embodiment of the invention in the form of a balloon drain. Unlike in the first embodiment, the fluid-carrying element 2 here is thrust displaceably onto a puncture stylet 5. The tip 5a of the stylet 5a is sharp. The tube 2d of the fluid-carrying element 2 ends in a distal wall portion 2c of an open-pore material 3 for draining fluid. An inflated annular balloon 1, which can be inflated via a channel 1b, is seated on the tube 2d.

FIG. 2b is a longitudinal sectional view of the fourth embodiment. The puncture stylet 5 can be seen with its sharp tip 5a. The tube ends in an open-pore wall portion 2c. The inflated balloon 1 communicates fluidically with the channel 1b for filling the balloon 1. The channel 1b is integrated with the wall of the tube 2d.

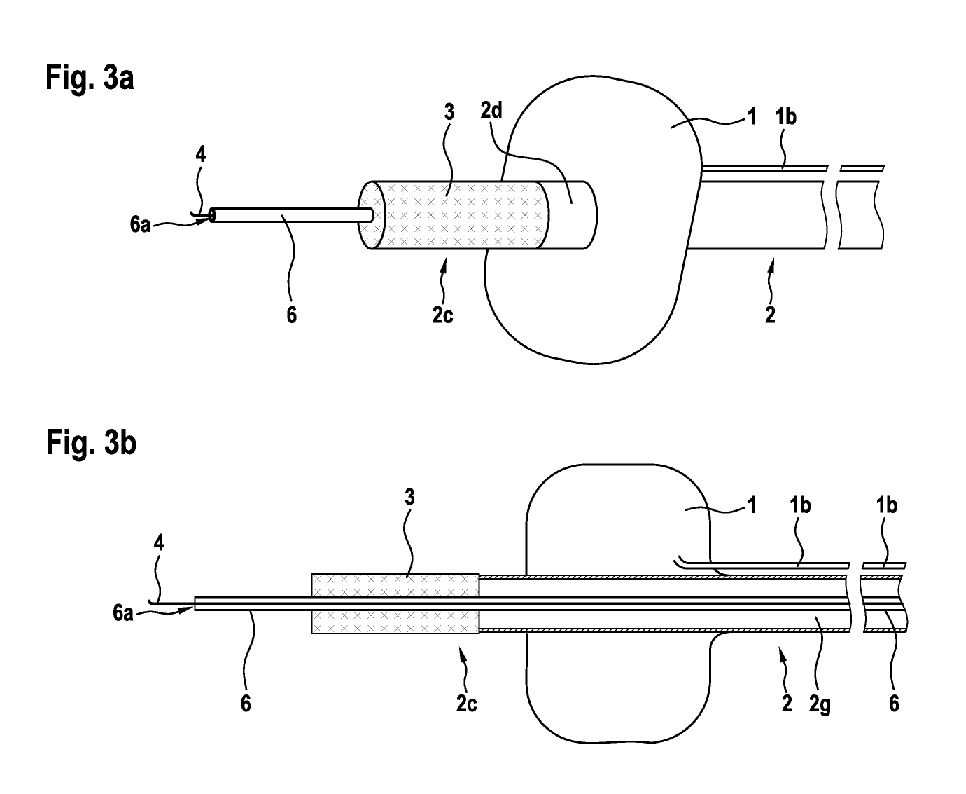

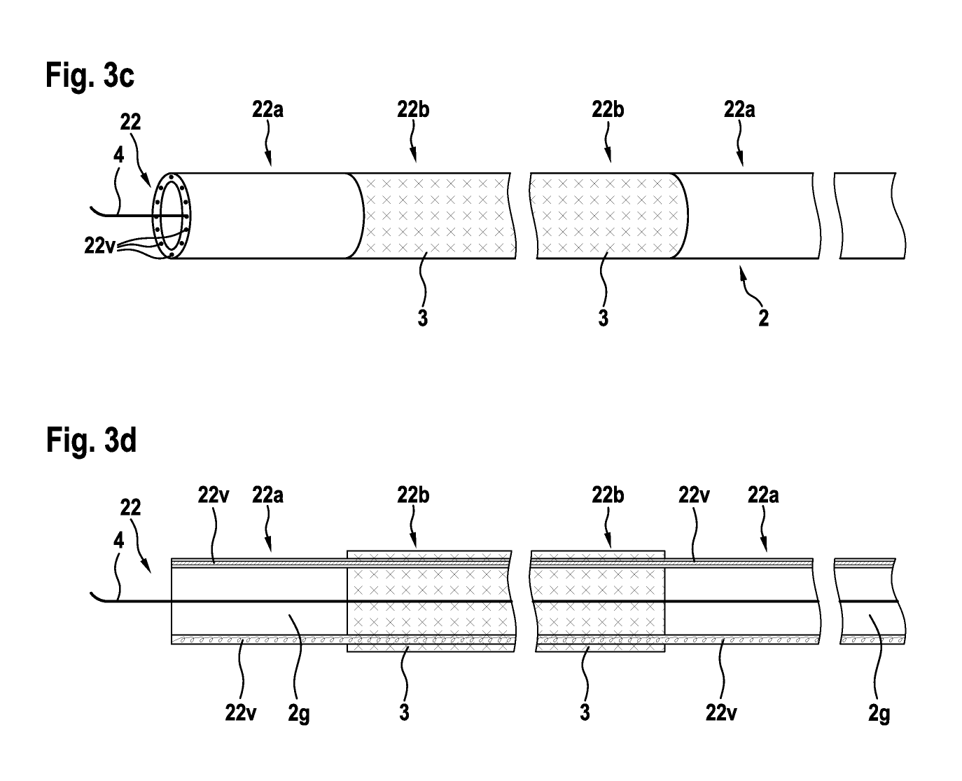

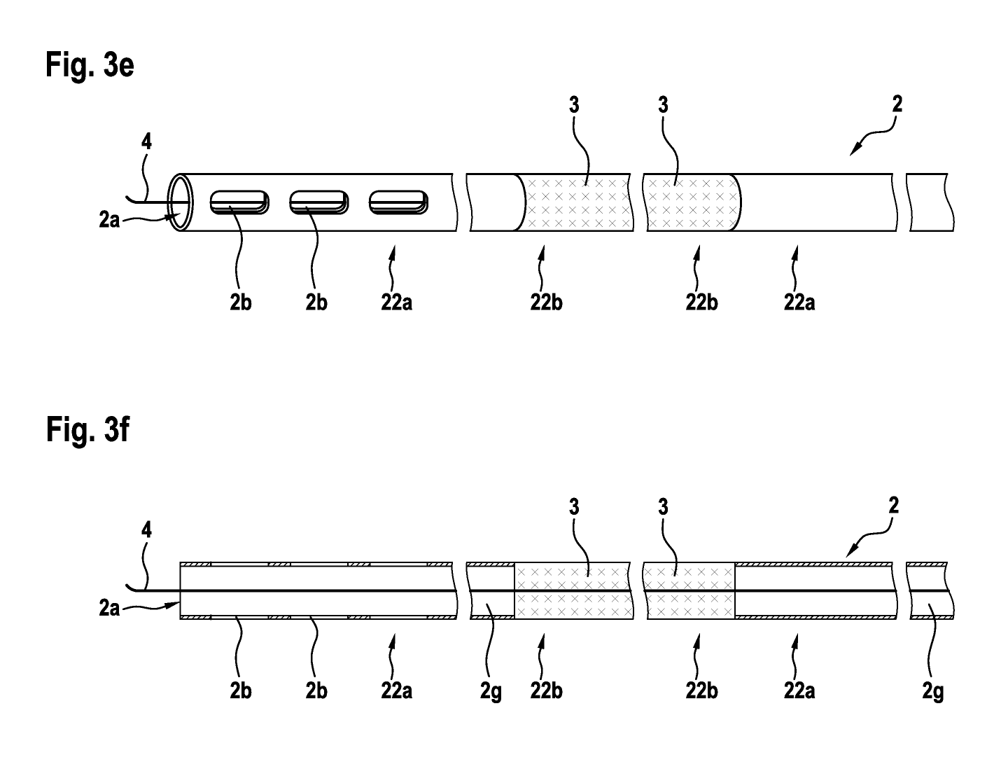

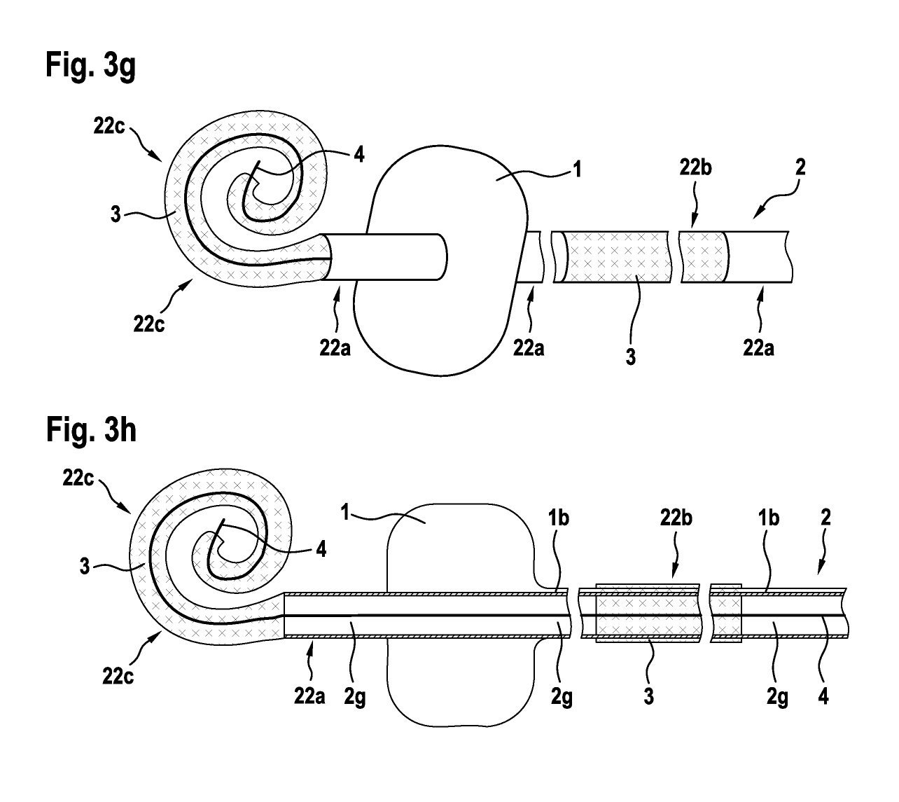

FIG. 3a is a side view of a fifth embodiment of the invention in the form of a balloon drain. The expanded balloon 1 can be seen, which communicates with a channel 1b in the form of a tube for filling the balloon 1. The fluid-carrying element 2 is spaced apart from the channel 1b for filling the balloon. The drainage tube 2d of the fluid-carrying element 2 ends distally in an open-pore wall portion 2c. Centrally in the inner lumen 2g of the fluid-carrying element 2, a rinsing channel 6 in the form of a further tube is inserted, from the distal opening 6a of which a guide wire 4 protrudes. Liquid can be delivered via the rinsing channel 6. The negative pressure is applied to the proximal end of the fluid-carrying element 2.

FIG. 3b is a longitudinal sectional view of the fifth embodiment. The expanded balloon 1 communicates fluidically with a channel 1b, which is spaced apart from the fluid-carrying element 2. The rinsing channel 6 in the form of a further tube for rinsing extends within the inner lumen 2g. A guide wire 4 protrudes from the tube end 6a.

In FIGS. 3c and 3d, a detail view of part of a fluid-carrying element of a sixth embodiment of a catheter of the invention is shown in a side view (FIG. 3c) and in a longitudinal sectional view (FIG. 3d).

The side view of FIG. 3c shows the fluid-carrying element 2, which comprises tubelike portions 22a and open-pore portions 22b. The open-pore portion 22b entirely comprises an open-pore material 3. A guide wire 4 protrudes from the proximal end of the fluid-carrying element 2. On the distal end 22 of the tube, reinforcing elements 22v can be seen in the tube wall.

In the longitudinal sectional view of FIG. 3d, the tubelike drain portions 22a and an open-pore drain portion 22b can be seen. These are reinforced in their walls by spiral and straight threadlike elements 22v. The tubelike portion 22a has an inner lumen 2g, which communicates fluidically with the open-pore material 3. In the vicinity of the open-pore portion 22b, the fluid-carrying element 2 itself does not have any fluid-carrying channel-like inner lumen that extends in the longitudinal axis of the drain. The fluid is carried along the open pores. A guide wire 4 is passed through the open-pore material 3 and through the inner lumen 2g of the tubelike portion 22a.

In FIGS. 3e and 3f, a detail view of part of a fluid-carrying element 2 of a seventh embodiment of a catheter of the invention is shown in a side view (FIG. 3e) and in a longitudinal sectional view (FIG. 3f).

A drainage tube 22a has a distal tube opening 2a and lateral perforation openings 2b; a middle portion of the fluid-carrying element 2 comprises an open-pore portion 22b, which comprises an open-pore material 3. A guide wire 4 is introduced into the catheter.

FIG. 3f is a longitudinal sectional view of the seventh embodiment. The distal tube portion can be seen, with lateral perforation openings 2b and an inner lumen 2g. The middle portion 22b comprises an open-pore material 3, which is not equipped with a channel or inner lumen along the main axis of the fluid-carrying element 2, but communicates fluidically with the inner lumen 2g of the drainage tube 22a. The fluid is carried along the open pores that communice with one another. The guide wire 4 extending in the inner lumen 2g and through the open-pore material 3 protrudes from the distal opening 2a of the tube.

In FIGS. 3g and 3h, an eighth embodiment of a catheter of the invention, in the form of a balloon drain, is shown in a side view (FIG. 3g) and in a longitudinal sectional view (FIG. 3h). The drain is shown with an expansion element in the form of an expanded balloon 1, whose cross section is increased for firmly holding the distal catheter in a hollow organ. The balloon 1 is seated annularly on a fluid-carrying element 2, which has a tubelike portion 22a with an inner lumen 2g, which extends from the proximal end, emerging from the body and shown in the drawings on the right, in the direction of the distal end of the catheter, to be located in the body interior and shown on the left in the drawing. The balloon 1 can be inflated via a channel 1b.

The distal end 22c of the tube comprises an open-pore material 3; it does not itself have any inner lumen, and the fluid carrying takes place along the open pores. A guide wire 4 protrudes from the distal end 22c. The distal end 22c is crimped in pigtail-like fashion.

A middle portion 22b of the fluid-carrying element 2 also comprises an open-pore material 3 and in that portion has no fluid-carrying inner lumen along the longitudinal axis; the channel 1b for filling the balloon 1 can be seen.

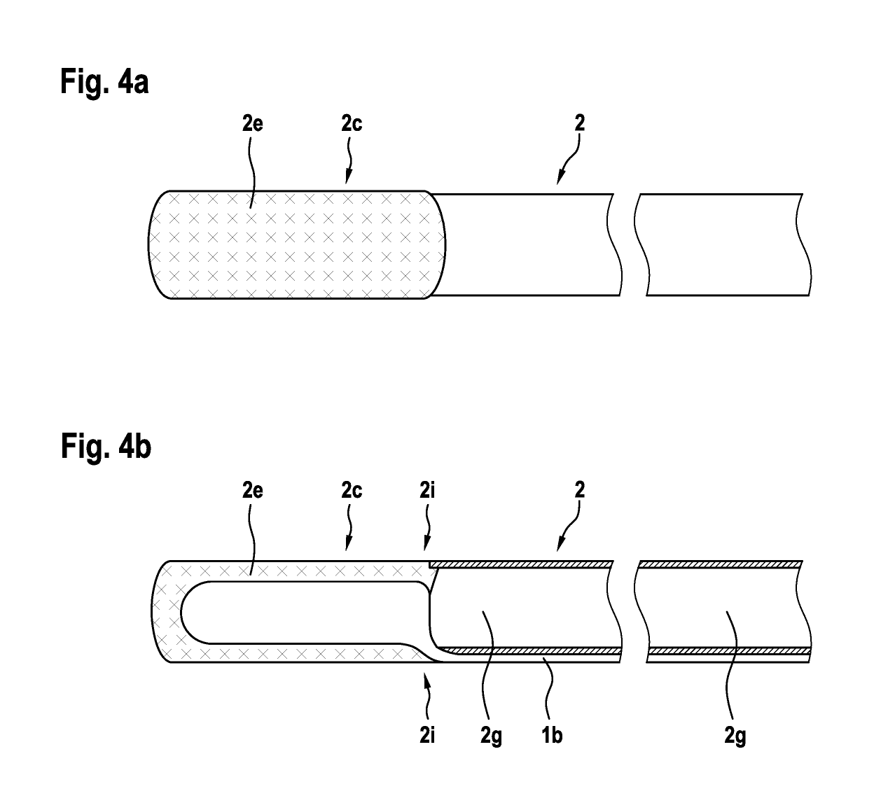

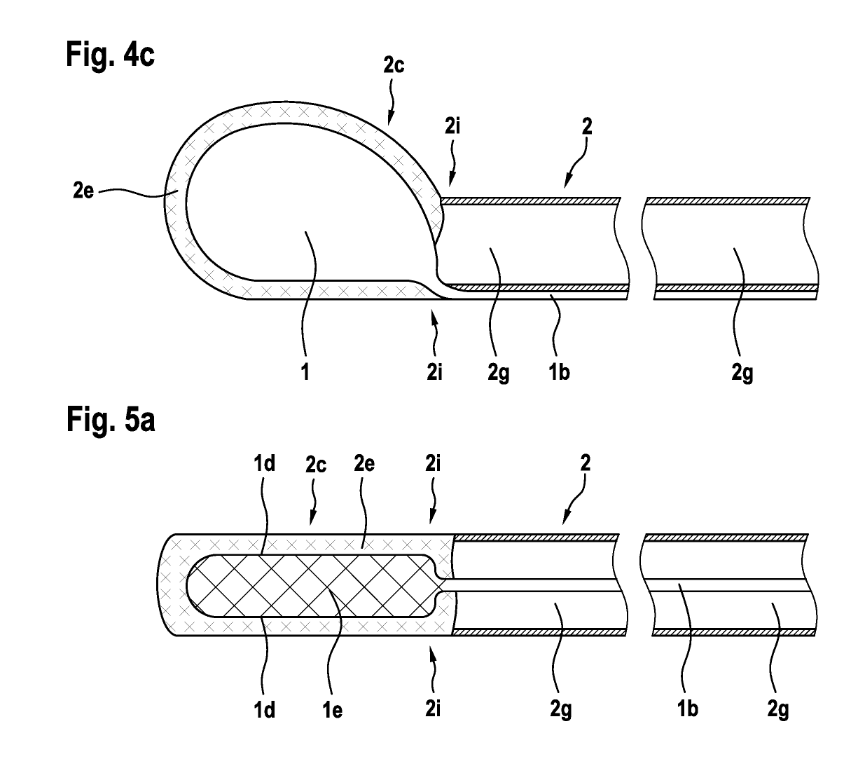

FIG. 4a is a side view of a ninth embodiment of the invention in the form of a balloon drain. The balloon 1 in this view has not yet unfurled but instead is collapsed. The fluid-carrying element 2 can be seen, with the drainage tube that ends in open-pore fashion on its distal end 2e. The distal front of the catheter here ends atraumatically in rounded fashion.

FIG. 4b is a longitudinal sectional view of the ninth embodiment. A channel 1b is integrated with the side wall of the fluid-carrying element 2 and serves to fill the balloon 1, which extends cylindrically in the distal end 2e of the drainage tube. The stretchable outer wall of the collapsed balloon 1 is sheathed by the open-pore wall 2c, which is capable of stretching elastically with the balloon 1 and in the process continues to remain fluid-carrying in open-pore fashion. Under a negative pressure, liquids or gases are carried through the open-pore wall portion 2c into the inner lumen 2g of the tube. The wall of the drainage tube merges at 2i with the open-pore wall portion 2c.

FIG. 4c is a longitudinal sectional view of the ninth embodiment, in which the balloon 1 is expanded. The channel 1b, which serves to fill the balloon 1, is integrated with the side wall of the fluid-carrying element 2. The open-pore wall 2c on the distal end 2e of the fluid-carrying element 2 stretches with the balloon 1 and in so doing continues to maintain the open-pore fluid carrying for gases and liquids. Under a negative pressure, liquids or gases are carried through the open-pore wall 2c into the inner lumen 2g of the tube.

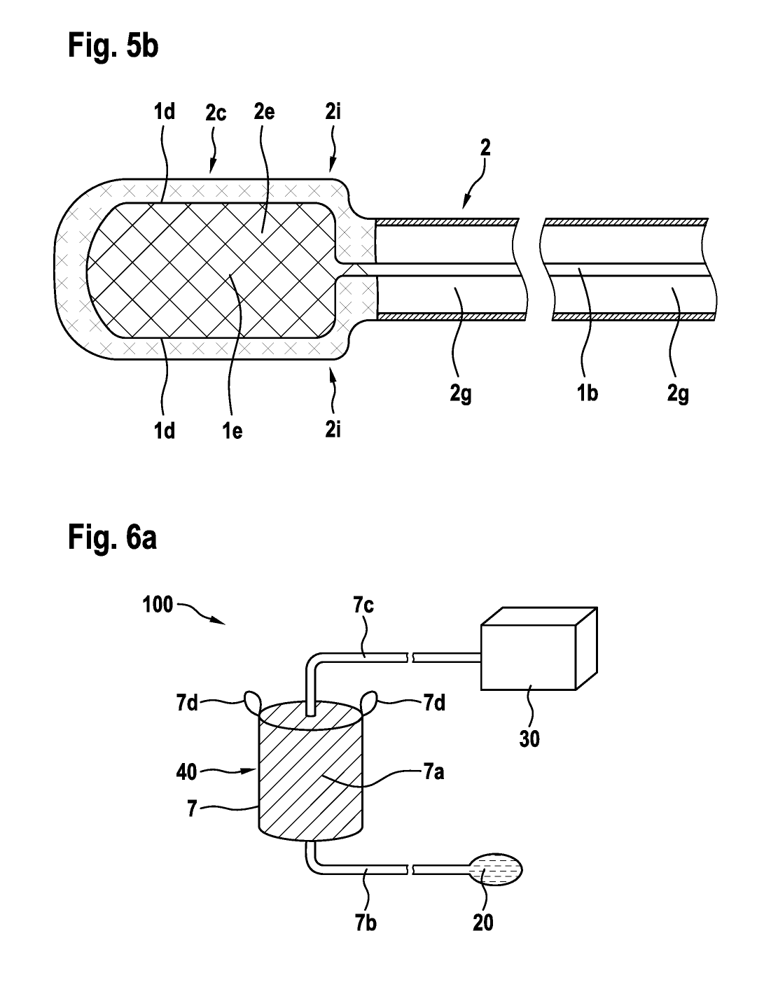

FIG. 5a is a longitudinal sectional view of a tenth embodiment of the invention in the form of a balloon drain. The tube portion of the fluid-carrying element 2 ends distally in the open-pore wall portion 2c. The open-pore wall portion 2c surrounds the balloon 1, which is filled with an elastically compressible filler material 1e (for instance, an open-pore polyurethane foam). The elastic balloon wall, on the outside of which the wall portion 2c is located, is shown at 1d. Via the channel 1b, a negative pressure has been applied to the balloon 1, so that the filler material 1e is compressed. The tube portion of the fluid-carrying element 2 merges at 2i with the open-pore wall portion 2c. The fluid-carrying inner lumen is shown at 2g.

FIG. 5b is a longitudinal sectional view of this balloon drain. Here, however, no negative pressure is applied to the channel 1b; instead, a normal air pressure is located there. The compressible filler material 1e has therefore unfurled and elongated with the balloon wall 1d and the open-pore wall portion 2c. In this way, a balloon drain placed in a cavity can adapt to the cavity with gentle stretching pressure, so that as much open-pore drainage area as possible contacts the body and can be pressed by suction two-dimensionally against it.

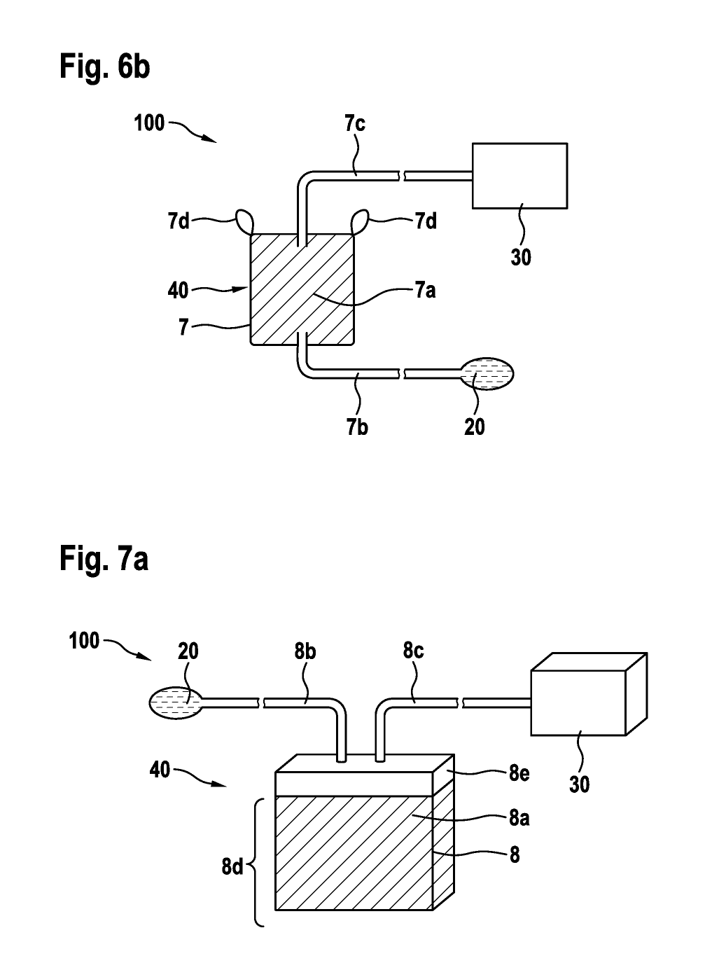

FIG. 6a shows a side view of a catheter system 100 of the invention, and FIG. 6b shows the catheter system 100 in a sectional view. The catheter system 100 has the following: a fluid collection container 40 in the form of a drainage bag 7, a negative-pressure generating system 30 in the form of an electronic pump, and a catheter 20 of the invention (merely suggested in the drawing). The drainage bag 7 is filled with a compressible open-pore filler material 7a. Via a first tube 7b, the bag 7 communicates with the catheter 20; via a second tube 7c, the bag 7 communicates with the pump. The bag 7 can be hung up by two loops 7d.

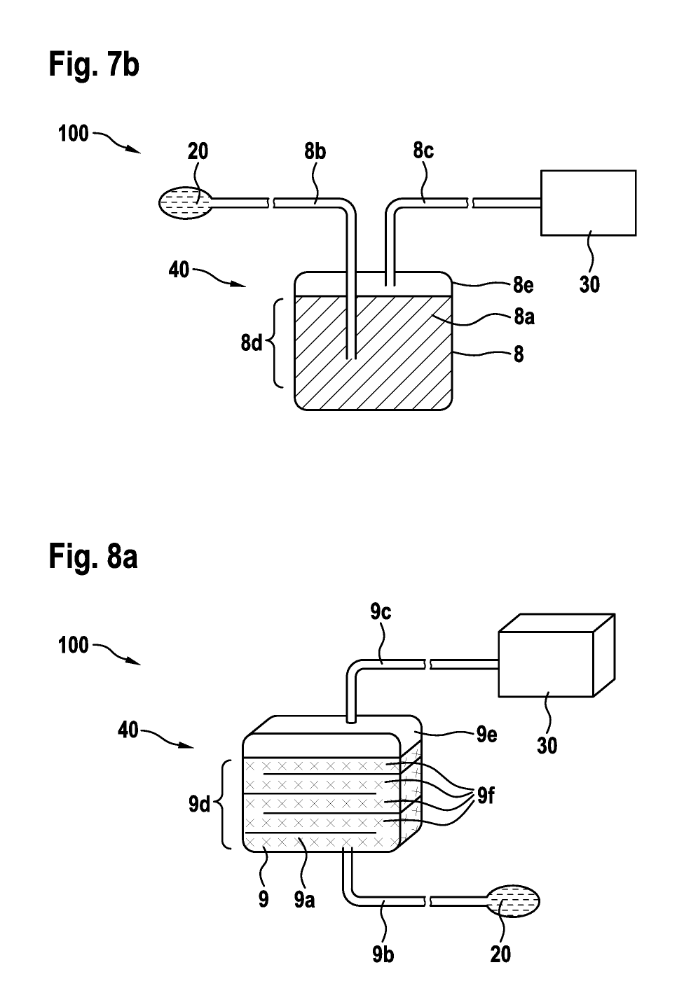

FIG. 7a shows a side view of an alternative embodiment of a catheter system of the invention, and FIG. 7b shows this catheter system in a sectional view. The catheter system has the following: a fluid collection container 40, a negative-pressure generating system 30 in the form of an electronic vacuum pump, and a catheter 20 of the invention. The fluid collection container 40 is embodied in the form of a secretion collection vessel 8, which comprises a solid canister part 8e and a flexible bag part 8d. In the bag part 8d, there is an open-pore compressible filler material 8a, such as an open-pore polyurethane foam. Via a first tube 8b, the secretion collection vessel 8 communicates with the catheter 20. The secretion collection vessel 8 communicates with the electronic vacuum pump 30 via a second tube 8c. In FIG. 7b, it is shown that the first tube 8b reaches to deep within the filler material 8a, while the second tube 8c reaches only into the solid-wall, negative pressure-stable canister part 8e.

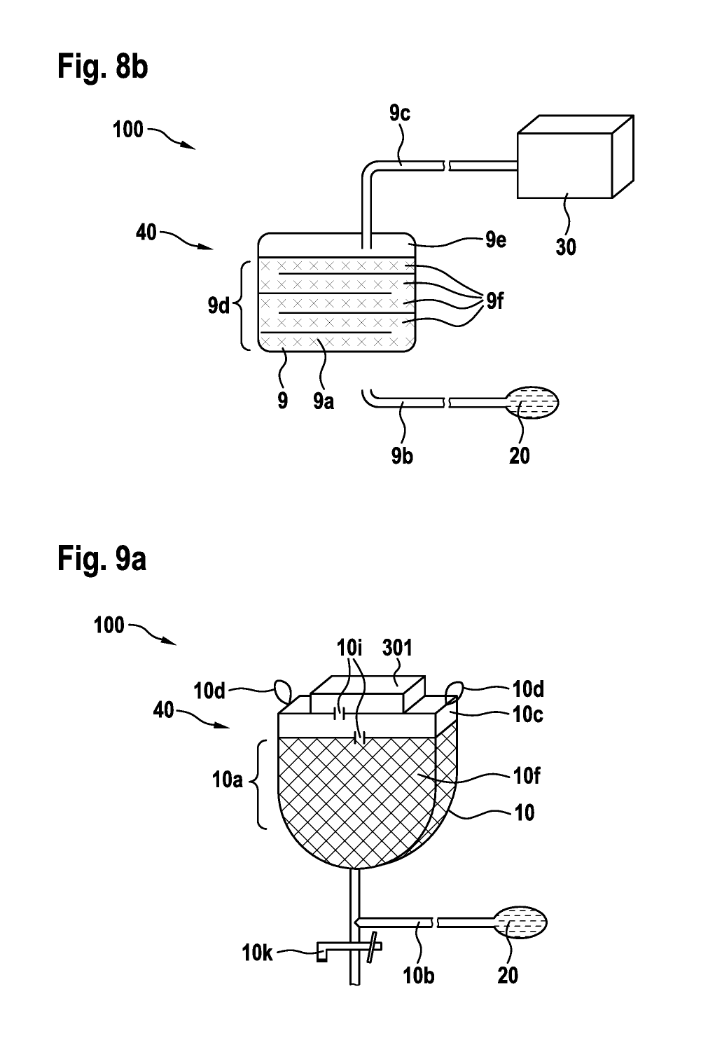

FIG. 8a shows a side view of a further alternative embodiment of a catheter system of the invention, and FIG. 8b shows this catheter system in a sectional view. The catheter system has the following: a fluid collection container 40, a negative-pressure generating system 30 in the form of an electronic vacuum pump, and a catheter 20 of the invention. The fluid collection container 40 is embodied in the form of a secretion collection vessel 9, which comprises a solid canister part 9e and a flexible bag part 9d. In the bag part 9d, there is an open-pore compressible filler material 9a, such as an open-pore polyurethane foam. The bag part 9d is divided into individual chambers 9f, which communicate fluidically with one another in meandering fashion. Via a first tube 9b, the secretion collection vessel 9 communicates with the catheter 20. The secretion collection vessel 9 communicates with the vacuum pump 30 via a second tube 9c.

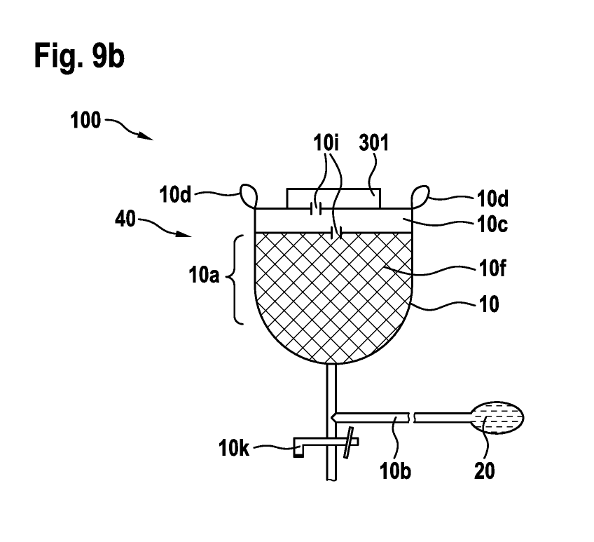

FIG. 9a shows a side view of a further alternative embodiment of a catheter system of the invention, and FIG. 9b shows this catheter system in a sectional view. The catheter system has the following: a fluid collection container 40, a negative-pressure generating system 30 in the form of a vacuum pump 301, and a catheter 20 of the invention. The fluid collection container 40 is embodied in the form of a secretion collection vessel 10, which comprises a solid canister part 10c and a flexible bag part 10a. In the bag part 10a, there is an open-pore compressible filler material 10f, such as an open-pore polyurethane foam. Via a first tube 10b, the secretion collection vessel 10 communicates with the catheter 20. The vacuum pump 301 communicates fluidically, as an integrated, removable part of the secretion collection vessel 10, with that secretion collection vessel in releasably locked fashion. The fluid or the negative pressure is carried via valves 10i from the pump 301 via the canister part 10c. Reference numeral 10k indicates an outlet stub with a tap; when the tap is opened, the secretion collected in the secretion collection vessel 10 can drain out.

Each of the catheter systems shown in FIGS. 6 through 10 can be equipped with an arbitrary catheter of the invention, in particular with one of the balloon catheters that are shown in FIGS. 1 through 5. The catheter of the invention is not limited, however, to a balloon catheter; instead of a balloon, it may also have some other expansion element. What is important is that the fluid-carrying element of the catheter has one or more wall portions of an open-pore material, such as a foam or an open-pore film assembly, and a negative pressure can be applied to the inner lumen of the fluid-carrying element.

The invention is not limited to the embodiments described above in detail and as shown as examples in the drawings, and the features explained in the foregoing description may be employed individually or in arbitrary combination with a catheter. Moreover, a catheter of the invention can advantageously, but not necessarily, be used for negative pressure therapy. Accordingly, embodiments of a catheter of the invention can be used even without a negative-pressure generating system.

What is claimed and desired to be secured by Letters Patent is as follows:

1. A catheter, in particular a balloon catheter,
having a fluid-carrying element with an inner lumen for carrying away body fluids and/or gases from the interior of the body, and
having an expansion element, such as a balloon, of variable cross section for keeping the catheter portion in a body cavity, characterized in that the fluid-carrying element has at least one wall portion of an open-pore material, which communicates fluidically with the inner lumen and through which the body fluid can be aspirated into the inner lumen by applying a negative pressure to a proximal end of the fluid-carrying element.

2. The catheter of claim 1, characterized in that the open-pore material has many pores communicating with one another in fluid carrying fashion.

3. The catheter of claim 2, characterized in that the pores have a diameter between 100 μm and 2000 μm, in particular between 100 μm and 500 μm, and/or that the mean spacing between the adjacent pores is between 100 μm and 5000 μm, in particular between 100 μm and 1000 μm.

4. The catheter of one of the foregoing claims, characterized in that the open-pore material is embodied in the form of an open-pore foam, such as a polyurethane foam, or in the form of an open-pore single-, double-, or multi-layer film.

5. The catheter of one of the foregoing claims, characterized in that the inner lumen is demarcated at least in some portions directly by the open-pore material, or alternatively that the inner lumen is bounded by a side wall having openings, and the openings are at least partly covered on the outside by the open-pore material.

6. The catheter of one of the foregoing claims, characterized in that the open-pore material is open-pored on one side, and the open-pore side faces outward for contact with a body tissue, and the inner side opposite from the open-pore side faces toward the inner lumen and has apertures.

7. The catheter of one of the foregoing claims, characterized in that the open-pore material has a nontransparent, in particular colored, film.

8. The catheter of one of the foregoing claims, characterized in that the wall portion of open-pore material is located in a distal end portion of the fluid-carrying element.

9. The catheter of one of the foregoing claims, characterized in that the expansion element is a balloon having an elastically stretchable wall, the wall being at least partly bounded by a fillable volume of the balloon.

10. The catheter of claim 9, characterized in that the fillable volume of the balloon is at least partly filled by a filler material, such as an open-pore polyurethane foam, which contracts when a negative pressure is applied to the fillable volume and causes the balloon to unfurle when the negative pressure is discontinued.

11. The catheter of claim 9 or 10, characterized by a fluid and/or gas carrying conduit for filling or emptying the fillable volume, which conduit extends essentially parallel to the fluid-carrying element.

12. The catheter of claim 11, characterized in that the conduit extends at least in some portions in the inner lumen of the fluid-carrying element, outside the inner lumen of the fluid-carrying element, in or contacting a side wall of the fluid-carrying element in its longitudinal direction, or alternatively extends as a separate tube spaced apart from the fluid-carrying element.

13. The catheter of one of claims 9 through 12, characterized in that the wall portion of the open-pore material adjoins the balloon distally, or alternatively that the balloon forms the distal end of the catheter, and the wall portion of the open-pore material is located at least in some portions on the outside of the elastically stretchable wall of the balloon.

14. The catheter of one of the foregoing claims, characterized in that a distal end of the catheter is embodied in closed fashion, and has a fastening element, such as an eyelet, a thread, a ball, or the like for attaching a grasping or tugging element.

15. The catheter of one of claims 1 through 13, characterized in that a distal end of the catheter has an opening, through which a guide wire or guide stylet can be inserted.

16. The catheter of one of the foregoing claims, characterized in that a distal end of the catheter has a cutting edge or tip for puncturing tissue.

17. The catheter of one of the foregoing claims, characterized in that the catheter has at least one rinsing channel, which on its distal end has an opening, for rinsing or supplying liquids, medications, or measuring instruments to a body cavity.

18. A catheter system having a catheter of one of the foregoing claims and a negative-pressure generating system, such as a pump system, which for generating suction in the inner lumen of the fluid-carrying element is attachable to the proximal end of the fluid-carrying element.

19. The catheter system of one of the foregoing claims, characterized by a negative-pressure-stable fluid collection container, connected between the negative-pressure generating system and the fluid-carrying element, such as an envelope and/or canister system.

20. The catheter system of one of the foregoing claims, characterized in that the negative-pressure generating system is arranged for generating a permanent negative pressure between approximately 40 mmHg and approximately 100 mmHg, in particular between approximately 20 mmHg and approximately 200 mmHg.
